(19) United States
(12) Patent Application Publication
(10) Pub. No.: US 2024/0285496 A1
(43) Pub. Date: Aug. 29, 2024

(54) Silicone-Free Hair Conditioner

(71) Applicant: Henkel AG & Co. KGaA, Duesseldorf (DE)

(72) Inventors: Manuela Mette, Schönberg OT Kleinfeld (DE); Thomas Schroeder, Hamburg (DE); Petra Westphal, Neu Wulmstorf (DE); Soeren Scheele, Hamburg (DE)

(21) Appl. No.: 18/660,711

(22) Filed: May 10, 2024

Related U.S. Application Data
(63) Continuation of application No. PCT/EP2022/079509, filed on Oct. 24, 2022.

(30) Foreign Application Priority Data
Nov. 24, 2021 (DE) 102021213218.9

(51) Int. Cl. A61K 8/46; A61K 8/37; A61K 8/42; A61K 8/44; A61Q 5/12

(52) U.S. Cl. CPC A61K 8/463; A61K 8/375; A61K 8/42; A61K 8/44; A61Q 5/12; A61K 2800/34

(57) ABSTRACT

Silicone-free hair care compositions contain trimethylglycine, esterquats, and mono-, di- and triglycerides of vegetable oils in a cosmetic carrier, which are suitable for improving the care properties of hair, in particular for improving the sensory properties as well as the resistance of hair to physical damage.

### FIELD OF THE INVENTION

The present application describes silicone-free hair care compositions containing trimethylglycine, an esterquat and mono-, di- and triglycerides of vegetable oils in a cosmetic carrier, and the use thereof for improving hair care.

### BACKGROUND OF THE INVENTION

Due to the heavy stressing of the hair, for instance coloring or permanent waves, frequent cleaning with shampoos and subsequent drying with blow-drying heat, and environmental stresses, the importance of hair care products is steadily increasing. At the same time, the subject of sustainability is increasingly the focus of cosmetic manufacturers due to the growing demands of consumers in this regard.

Hair care products in a wide variety of forms are known per se and are commercially available. The majority of these products comprises silicones, because they impart a variety of conditioning advantages to hair. When providing sustainable products, the use of silicones is largely dispensed with, but this usually leads to disadvantages in the care performance of the products. Numerous attempts have been made to eliminate these drawbacks. For example, it has been tested whether the omission of silicones can be compensated for by an increase in the use concentration of other care ingredients. This was only partially successful, because while satisfactory or even good care results could be achieved on wet hair, the care performance of such products on dry hair was insufficient.

DE 102018202803 discloses hair conditioning products free of silicone and/or mineral oil containing trimethylglycine, amidoamines and specific esterquats, which give both wet and dry hair improved combability, detangling and gloss properties, as well as a softer hair feel.

Nevertheless, there still exists a need for natural cosmetic products having very good care capacity, in particular on dry hair, with which the resilience or resistance of hair to external influences can be improved. The achieved care effects should be at least comparable to products containing silicones.

### BRIEF SUMMARY OF THE INVENTION

These objects were achieved by providing a silicone-free hair care composition which contains in a cosmetic carrier, based on the total weight of the hair care composition:
a) 0.1-2.0 wt. % trimethylglycine (INCl name: Betaine) and
b) 0.05-8.0 wt. % of a mixture of
(b1) at least one esterquat of the following formula (II),

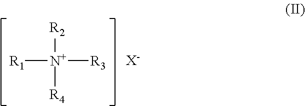

wherein
R₁, R₂ are identical or different and represent a C₁-C₄ alkyl radical,
R₃, R₄ are identical or different and represent the group —CH₂—CH(R₅)—OR₆, in which
R₅ represents a C₁-C₆ alkyl radical,
R₆ represents an acyl radical of an at least monounsaturated carboxylic acid having a C chain length of from 18 to 24 carbon atoms or represents the acyl radical of isostearic acid, and
X⁻ represents a halide, methyl sulfate, ethyl sulfate, maleate, fumarate, oxalate, tartrate, citrate, lactate or acetate ion, and
(b2) mono-, di- and triglycerides of vegetable oils.

The hair care compositions according to the invention ensure an excellent balance between hair care and protection of the hair fibers. In particular, due to the use of the hair care compositions according to the invention on wet and dry hair, it was possible to achieve excellent care effects, such as improved combability, hair feel and increased hair volume, without high amounts of active ingredient and/or the use of silicones being required.

The use of the hair care compositions according to the invention additionally made it possible to increase the resistance of hair to physical damage (for example caused by combing and/or blow drying).

The improved biodegradability compared to silicone-containing products is a further advantage of the hair care compositions according to the invention, because they contain a predominant portion of active ingredients from renewable sources.

Within the meaning of the present invention, a cosmetic carrier is understood to mean an aqueous, an alcoholic or an aqueous-alcoholic carrier. For the purpose of hair care, such carriers are understood, for example, to be creams, emulsions, gels, pastes, or also surfactant-containing foaming solutions, such as shampoos, foam aerosols, foam formulations, or other preparations which are suitable for application to the hair.

An aqueous carrier is preferred according to the invention and preferably comprises 50-95 wt. % water, based on the total hair care composition.

### DETAILED DESCRIPTION OF THE INVENTION

As a first essential ingredient, the hair care compositions according to the invention contain trimethylglycine (INCl name: Betaine). Trimethylglycine is an amino acid derivative which is well known for use in cosmetic compositions, and which is obtained from natural sources such as sugar beet molasses. Natural betaine is commercially available, for example, from Evonik under the name Tego® and is used as a humectant in preparations for skin treatment and as a strengthening active ingredient in hair care compositions.

It was observed that the hair care properties of specific esterquats b) can be further improved by adding trimethylglycine. In particular, the detangling and combability properties of dry hair, and the soft hair feel right to the hair tips can be improved by the combination of these active ingredients.

Trimethylglycine is used in the hair care compositions according to the invention (based on the total weight of the compositions) in an amount of from 0.1 to 2.0 wt. %, preferably from 0.2 to 1.9 wt. %, particularly from 0.25 to 1.75 wt. %, more particularly from 0.4 to 1.6 wt. %, and most particularly from 0.5 to 1.5 wt. %.

A second essential component of the care active ingredient mixture in the hair care compositions according to the invention is an esterquat (b1) of formula (II),

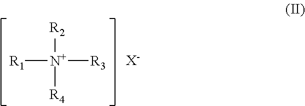

wherein
R₁, R₂ are identical or different and represent a C₁-C₄ alkyl radical,
R₃, R₄ are identical or different and represent the group —CH₂—CH(R₅)—OR₆, in which
R₅ represents a C₁-C₆ alkyl radical,
R₆ represents an acyl radical of an at least monounsaturated carboxylic acid having a C chain length of from 18 to 24 carbon atoms or represents the acyl radical of isostearic acid, and
X⁻ represents a halide, methyl sulfate, ethyl sulfate, maleate, fumarate, oxalate, tartrate, citrate, lactate or acetate ion.

Esterquats (b1) enhance the hair-conditioning effect within the active ingredient combination a)+b) and improve in particular the hair feel.

It was found that particularly good hair conditioning results can be achieved with esterquats (b1), which contain unsaturated acyl radicals R₆ of a specific C-chain length.

Particularly preferred esterquats (b1) are therefore selected from compounds of the formula (II) in which
R₁, R₂ are identical and represent a methyl radical,
R₃, R₄ are identical and represent the group —CH₂—CH(R₅)—OR₆, in which
R₅ represents a methyl radical,
R₆ represents the acyl radical of oleic acid, and
X⁻ represents a chloride or methyl sulfate ion.

Particular preference is given to esterquats (b1) according to formula (II) which are known under the name ethanaminium, N-(2-hydroxyethyl)-N-methyl-2-[1-oxo-9-octadecenyl)oxy]-N-[2-[(1-oxo-9-octadecenyl)oxy]ethyl]-methyl sulfate (salt) (INCl name: Dioleoylethyl Hydroxyethylmonium Methosulfate) and which are commercially available, for example, under the name Tetranyl® CO-40 from Kao.

Dioleylethyl Hydroxyethylmonium Methosulfate is an ecological and biodegradable cationic surfactant which gives hair fibers a soft feel and excellent antistatic properties. In combination with trimethylglycine and vegetable oil glycerides, it also improves the combability of wet and dry hair and protects hair fibers from blow-drying heat and against damage caused by mechanical effects.

The esterquat(s) (b1) is (are) used in the hair care compositions according to the invention (based on the total weight of the compositions) in an amount of from 0.01 to 6.0 wt. %, preferably from 0.02 to 5.5 wt. %, particularly from 0.03 to 5.0 wt. %, more particularly from 0.04 to 4.5 wt. % and most particularly from 0.05 to 4.0 wt. %.

Suitable mono-, di- and triglycerides of vegetable oils (b2) are preferably understood to mean mixtures of mono-, di- and triglycerides of sunflower oil and/or rapeseed oil, for example a glyceride mixture known under the INCl name Sunflower Seed Oil Glycerides.

Mono-, di- and triglycerides of vegetable oils (b2) can be used individually or as a mixture with the care active ingredients a) or (b1) in the hair care compositions according to the invention.

When used individually in the hair care compositions according to the invention, component (b2) is preferably used in an amount of from 0.01 to 3.0 wt. % with respect to the total weight of the hair care composition.

When used as a mixture, it may, for example, be used together with the esterquat (b1) in the hair care compositions according to the invention. Such mixtures are commercially known and available, for example under the trade name Stepanquat® Soleil.

The commercial product Stepanquat® Soleil can be used in the compositions according to the invention in an amount of from 0.05 to 8.0 wt. %, preferably from 0.25 to 7.0 wt. %, more preferably from 0.5 to 6.0 wt. %, particularly from 0.75 to 5.0 wt. %, and most particularly from 1.0 to 4.0 wt. %, the quantitative data being based on the total weight of the hair care compositions.

For further assistance or enhancement of the care and protective effect of the compositions according to the invention, it is advantageous if they also contain at least one amidoamine.

Within the meaning of the present invention, suitable amidoamines are understood to mean compounds of the following formula (I),

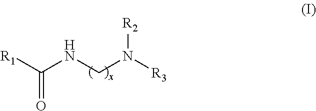

wherein
R₁ represents a saturated or unsaturated, branched or unbranched alk(en)yl radical having 19 to 30 carbon atoms,
R₂ and R₃ represent, independently of one another, hydrogen or a C₁-C₄ alkyl group, and
x represents an integer from 2 to 6.

Amidoamines of the formula (I) give hair an extremely soft, smooth feel without weighing it down. It has been found that in particular amidoamines having an alk(en)yl radical (R₁) comprising more than 19 C atoms, in combination with the active ingredient combination a)+b) according to the invention, additionally improve combability and are better suited than the amidoamines usually used in the prior art (alk(en)yl radicals having s 18 C atoms), in preventing an overconditioning effect (build-up).

Therefore, particularly well-suited amidoamines are those according to formula (I), in which
R₁ represents an alkyl radical having 20 to 24 carbon atoms,
R₂ and R₃ represent methyl groups, and
x represents the number 2, 3 or 4.

Particularly preferred for use in the hair care compositions according to the invention are amidoamines according to formula (I) which are selected from amidoamines c) known under the INCl name Brassicamidopropyl Dimethylamine.

Such amidoamines are commercially available from various suppliers and are particularly suitable for use in the hair care compositions according to the invention, for example Kerabase® LC from Inolex Personal Care Ingredients, ProCondition® 22 from Inolex Personal Care Ingredients and/or Amidet® APA-22 from Kao Corporation.

The amidoamine(s) is (are) preferably used in the hair care compositions according to the invention (based on the weight of the total composition) in an amount of from 0.1 to 3.0 wt. %, preferably from 0.2 to 2.5 wt. %, more preferably from 0.25 to 2.0 wt. %, particularly from 0.3 to 1.5 wt. %, and most particularly from 0.4 to 1.0 wt. % (OK).

In a particularly preferred embodiment, silicone-free hair care compositions according to the invention contain in a cosmetic carrier, based on their total weight:
a) 0.1-2.0 wt. % trimethylglycine (INCl name: Betaine),
b) 0.05-8.0 wt. % of a mixture of
(b1) at least one esterquat of the following formula (II),

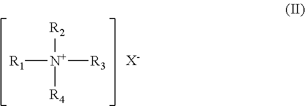

wherein
R₁, R₂ are identical or different and represent a C₁-C₄ alkyl radical,
R₃, R₄ are identical or different and represent the group —CH₂—CH(R₅)—OR₆, in which
R₅ represents a C₁-C₆ alkyl radical,
R₆ represents an acyl radical of an at least monounsaturated carboxylic acid having a C chain length of from 18 to 24 carbon atoms or represents the acyl radical of isostearic acid, and
X⁻ represents a halide, methyl sulfate, ethyl sulfate, maleate, fumarate, oxalate, tartrate, citrate, lactate or acetate ion, and
(b2) mono-, di- and triglycerides of vegetable oils and
c) 0.1-3.0 wt. % of at least one amidoamine of the following formula (I),

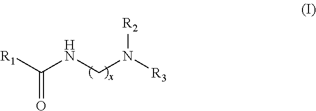

wherein
R₁ represents a saturated or unsaturated, branched or unbranched alk(en)yl radical having 19 to 30 carbon atoms,
R₂ and R₃ represent, independently of one another, hydrogen or a C₁-C₄ alkyl group, and
x represents an integer from 2 to 6.

Within this embodiment, particular preference is given to silicone-free hair care compositions which contain, based on their total weight:
a) 0.1-2.0 wt. % trimethylglycine (INCl name: Betaine),
b) 0.05-8.0 wt. % of a mixture of
(b1) at least one esterquat known under the INCl name Dioleylethyl Hydroxyethylmonium Methosulfate and
(b2) at least one glyceride mixture known under the INCl name Sunflower Seed Oil Glycerides, and
c) 0.1-3.0 wt. % of at least one amidoamine known under the INCl name Brassicamidopropyl Dimethylamine.

In order to further increase the care properties of the compositions according to the invention, it can be advantageous if they contain further active ingredients and auxiliary substances which give you advantageous properties.

In a further preferred embodiment, hair care compositions according to the invention therefore contain at least one further conditioning active ingredient selected from
triglycerides having a chain length of 6 to 14 carbon atoms,
isopropyl esters having a chain length of 10 to 20 carbon atoms,
vegetable oils,
glycerol
or mixtures thereof.

In order to further support the care effect of the active ingredients a)+b) (and optionally+c)) of the hair care composition according to the invention, the hair care composition according to the invention can further contain at least one compound selected from the group consisting of medium-chain triglycerides having a chain length of 6 to 14 carbon atoms.

Preference is given to medium-chain triglycerides derived from saturated fatty acids having 8 to 10 carbon atoms, in particular mixtures of caprylic/capric triglycerides. Suitable caprylic/capric triglycerides are available, for example, under the name Myritol® 318 from BASF SE.

Triglycerides having a chain length of 6 to 14 carbon atoms can be used in the hair care compositions according to the invention-based on their total weight-preferably in amounts from 0.1 to 5.0 wt. %, more preferably from 0.2 to 4.0 wt. %, and in particular from 0.25 to 3.0 wt. %.

Suitable isopropyl esters having a chain length of 10 to 20 carbon atoms are preferably understood to be isopropyl laurate, isopropyl myristate (for example the commercial product Rilanit® IPM), isopropyl palmitate (for example the commercial product Rilanit® IPP), isopropyl stearate, isopropyl isostearate, isopropyl oleate, and mixtures thereof.

Isopropyl myristate, isopropyl palmitate and mixtures thereof are particularly preferred.

The isopropyl ester or esters having a chain length of 10 to 20 carbon atoms is (are) used in the hair care compositions according to the invention (based on the weight of the total composition) preferably in an amount of from 0.1 to 5.0 wt. %, more preferably from 0.2 to 4.0 wt. %, and in particular from 0.25 to 3.0 wt. %.

For the purposes of the present invention, “vegetable oils” are understood to mean plant-based oil bodies which have a melting point of less than 50° C., particularly preferably less than 47° C., very particularly preferably less than 44° C., most preferably less than 40° C. Most preferred are cosmetic oils which are flowable at a temperature of less than 40° C.

Vegetable oils (and/or butters)—even used in low concentrations—can, in combination with the active ingredient combination a)+b) (and optionally+c)), further increase the aforementioned conditioning advantages on the hair, without a built-up effect occurring when regularly applied. In addition, vegetable oils (and/or butters) in the care active ingredient mixture of the hair care compositions according to the invention also contribute to improving hair gloss.

Examples of suitable natural vegetable oils according to the invention are, for example, amaranth seed oil, apricot kernel oil, argan oil, avocado oil, babassu oil, cottonseed oil, borage seed oil, camelina oil, canola oil, cranberry oil, thistle oil, peanut oil, pomegranate seed oil, grapefruit seed oil, hemp oil, rose hip seed oil, hazelnut oil, elderberry seed oil, blackcurrant seed oil, jojoba oil, cocoa butter, coconut oil, pumpkin seed oil, linseed oil, macadamia nut oil, corn oil, mallow oil, almond oil, mango seed oil, marula oil, poppy seed oil, evening primrose oil, olive oil, palm oil, palm kernel oil, peach kernel oil, rambutan oil, canola seed oil, rice bran oil, castor oil, sacha inchi oil, safflower oil, sea buckthorn fruit oil, sea buckthorn seed oil, sasanqua oil, sesame oil, shea butter, soybean oil, sunflower oil, tea tree oil, seed oil, grape seed oil, tsubaki oil, walnut oil, wheat germ oil, meadowfoam seed oil, and/or wild rose oil.

Preference is given to amaranth seed oil, apricot kernel oil, argan oil, avocado oil, coconut oil, almond oil, macadamia nut oil, rose hip seed oil, sunflower oil, olive oil, peach kernel oil, jojoba oil and/or the plant butters shea butter and/or cocoa butter.

The teaching according to the invention also includes the possibility of at least two natural, vegetable oils being mixed with one another.

The vegetable oil(s) (and/or butter(s)) is (are) used in the hair care compositions according to the invention (based on the weight of the total composition) in an amount of from 0.01 to 3.0 wt. %, preferably from 0.02 to 2.5 wt. %, more preferably from 0.025 to 2.0 wt. %, particularly from 0.04 to 1.5 wt. % and most particularly from 0.05 to 1.0 wt. %.

Glycerol as a further active ingredient can be used in the hair care compositions according to the invention (based on the weight of the total composition) preferably in an amount of from 0.1 to 15.0 wt. %, more preferably from 0.25 to 12.5 wt. %, particularly from 0.5 to 10.0 wt. %, more particularly from 0.75 to 7.5 wt. %, and most particularly from 1.0 to 5.0 wt. %.

The hair care compositions according to the invention can optionally contain one or more emulsifier(s) for solubilizing the aforementioned oils. Suitable emulsifiers are preferably understood to mean non-ionic polyethoxylated emulsifiers, particularly preferably ethoxylated fatty alcohols having 10 to 24 carbon atoms and/or ethoxylated castor oil.

Suitable ethoxylated fatty alcohols within the meaning of the invention are addition products of ethylene oxide to a fatty alcohol, wherein the degree of ethoxylation indicates the molar amount of ethylene oxide (EO) which was added on average per mole of fatty alcohol. Preferred ethoxylated fatty alcohols are addition products of ethylene oxide to capric alcohol, lauryl alcohol, isotridecyl alcohol, myristyl alcohol, cetyl alcohol, palmoleyl alcohol, stearyl alcohol, oleyl alcohol, elaidyl alcohol, petroselinyl alcohol, linoleyl alcohol, linolenyl alcohol, eleostearyl alcohol, arachyl alcohol, gadoleyl alcohol, behenyl alcohol, erucyl alcohol and brassidyl alcohol, and the technical mixtures thereof, which are obtained, for example, in the high-pressure hydrogenation of technical methyl esters based on fats and oils or aldehydes from Roelen's oxosynthesis and as a monomer fraction in the dimerization of unsaturated fatty alcohols. Particular preference is given to addition products to technical fatty alcohols or mixtures thereof having 12 to 18 carbon atoms, for example coconut, palm, palm kernel or tallow fatty alcohol, in particular coconut and/or tallow fatty alcohol.

Depending on the production method, the ethoxylated fatty alcohols are obtained as a mixture with differing distribution of degrees of ethoxylation. Within the meaning of the invention, these emulsifiers are therefore characterized according to the average degree of ethoxylation. This is usually discernible as a number after the fatty alcohol suffix “eth-” in the INCl name. Particularly suitable ethoxylated fatty alcohols are fatty alcohols with a degree of ethoxylation from 10 to 100, preferably 20 to 80 mol of ethylene oxide per mole of fatty alcohol. Examples are Ceteareth-12, Ceteth-15, Ceteareth-15, Laneth-16, Ceteth-16, Oleth-16, Steareth-16, Oleth-20, Ceteth-20, Ceteareth-20, Ceteareth-23, Laureth-23, Ceteareth-25, Ceteareth-30, Ceteth-40, Laneth-40, Oeth-50, Ceteareth-50, Ceteareth-60, and/or Ceteareth-80.

Suitable ethoxylated castor oil is preferably understood to mean ethoxylated, hardened (i.e., hydrogenated) and unhardened castor oil. The degree of ethoxylation indicates the molar amount of ethylene oxide (EO) which was added on average per mole of castor oil. Preferred ethoxylated castor oils are the compounds known under the following INCl names: PEG-5 Castor Oil, PEG-7 Hydrogenated Castor Oil, PEG-10 Hydrogenated Castor Oil, PEG-25 Hydrogenated Castor Oil, PEG-35 Castor Oil, PEG-36 Castor Oil, PEG-40 Castor Oil, PEG-40 Hydrogenated Castor Oil, PEG-50 Hydrogenated Castor Oil, PEG-60 Castor Oil, PEG-60 Hydrogenated Castor Oil, PEG-80 Castor Oil, PEG-80 Hydrogenated Castor Oil, PEG-100 Castor Oil, PEG-100 Hydrogenated Castor Oil, PEG-120 Castor Oil, PEG-120 Hydrogenated Castor Oil, PEG-150 Castor Oil, PEG-150 Hydrogenated Castor Oil, PEG-200 Castor Oil, PEG-200 Hydrogenated Castor Oil. Preferred non-ionic emulsifiers are castor oils having an average degree of ethoxylation of 20 to 150, preferably 30 to 100 and particularly preferably 35 to 80. PEG-40 Hydrogenated Castor Oil is particularly preferred.

From an ecological point of view, it is advantageous if the hair care compositions according to the invention contain the emulsifier(s)—if at all—in small amounts.

In a preferred embodiment, the hair care compositions according to the invention therefore preferably contain at most 1.0 wt. %, more preferably at most 0.8 wt. %, particularly preferably at most 0.6 wt. % and especially at most 0.5 wt. % of one or more non-ionic, ethoxylated emulsifiers (based on the total weight of the composition).

To further optimize the rheological properties of the hair care compositions according to the invention, these further active ingredients can contain fatty substances. Suitable fatty substances are to be understood as fatty acids and/or fatty alcohols which can be present both in solid form and in liquid form as an aqueous dispersion.

Linear and/or branched, saturated and/or unsaturated fatty acids having 6-30 carbon atoms can be used as suitable fatty acids. Preference is given to fatty acids having 10-22 carbon atoms. Among these, mention may be made, for example, of isostearic acid, such as the commercial products Emersol® 871 and Emersol® 875, and isopalmitic acid, such as the commercial product Edenor® IP 95, and also all other fatty acids sold under the Edenor® (Cognis) trade names. Other typical examples of such fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid and erucic acid, as well as the technical mixtures thereof. Particular preference is usually given to the fatty acid fractions which are obtained from coconut oil or palm oil; in particular, the use of stearic acid is generally preferred.

The amount of fatty acids used in the hair care compositions according to the invention is preferably 0.1-15 wt. %, based on the total composition. Particularly preferably, the amount of fatty acids used in the hair care compositions according to the invention is 0.5-10 wt. %, with amounts of 1-5 wt. % being very particularly advantageous.

As suitable fatty alcohols, use may be made of saturated, mono- or polyunsaturated, branched or unbranched fatty alcohols having 6 to 30, preferably 10 to 22 and very particularly preferably 12 to 20 carbon atoms. In the context of the invention, use may be made for example of decanol, octanol, octenol, dodecenol, decenol, octadienol, dodecadienol, decadienol, oleyl alcohol, erucic alcohol, ricinoleic alcohol, stearyl alcohol, isostearyl alcohol, cetyl alcohol, lauryl alcohol, myristyl alcohol, arachidyl alcohol, caprylic alcohol, capric alcohol, linoleyl alcohol, linolenyl alcohol and behenyl alcohol, and Guerbet alcohols thereof, this list being intended to be of exemplary and non-limiting nature.

In a preferred embodiment, the fatty alcohols are obtained from natural fatty acids, it being possible usually to start from an extraction from the esters of the fatty acids by reduction. Those fatty alcohol fractions which are a mixture of different fatty alcohols can also be used according to the invention. Such substances are commercially available, for example, under the names Stenol®, e.g. Stenol® 1618 or Lanette®, e.g. Lanette® O or Lorol®, e.g. Lorol® C8, Lorol® C14, Lorol® C18, Lorol® C8-18, HD-Ocenol®, Crodacol®, e.g. Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. Of course, lanolin alcohols, such as those commercially available under the names Corona®, White Swan®, Coronet® or Fluilan®, can also be used according to the invention.

The amount of fatty alcohols used in the hair care compositions according to the invention is preferably 0.1-20 wt. %, based on the total composition. Particularly preferably, the amount of fatty alcohols used in the hair care compositions according to the invention is 0.5-15 wt. %, with amounts of 1.0-10 wt. % being most particularly advantageous.

In a particularly preferred embodiment, the hair care compositions according to the invention contain at least one saturated or unsaturated, branched or unbranched fatty alcohol having 10 to 24 carbon atoms in an amount of from 1.0 to 10.0 wt. % (based on the weight of the total composition) for optimizing the rheological properties.

Within this embodiment, it is particularly preferred for the hair care compositions according to the invention to contain cetyl alcohol, stearyl alcohol and/or mixtures of cetyl alcohol and stearyl alcohol in the aforementioned amounts.

In a further preferred embodiment, the hair care compositions according to the invention can additionally contain at least one hydroxycarboxylic acid in a preferred amount (based on the weight of the total composition) of from 0.1 to 5.0 wt. %, more preferably from 0.2 to 4.0 wt. %, particularly from 0.3 to 3.0 wt. %, and most particularly from 0.4 to 2.0 wt. %, for adjusting the pH, but at the same time also for supporting the hair care properties.

Suitable hydroxycarboxylic acids are preferably understood to mean alpha-hydroxycarboxylic acids and in particular naturally occurring—for example in fruit acids-alpha-hydroxycarboxylic acids. These include, for example, malic acid, citric acid, glycolic acid, isocitric acid, mandelic acid, lactic acid, tartronic acid and/or tartaric acid. Suitable hydroxycarboxylic acids are also understood to mean the dicarboxylic acids glutamic acid and/or succinic acid. Particular preference is given to citric acid, lactic acid, glutamic acid and/or succinic acid.

Particularly good stability and particularly good hair care results (in particular with regard to hair gloss and sensory properties such as softness and smoothness) can be achieved without the occurrence of overconditioning (oily, weighed-down hair) if the hair care compositions according to the invention do not contain any further fatty phase components besides the aforementioned active ingredients.

The hair care compositions according to the invention can contain further optional active ingredients, provided that they do not impair the effectiveness or the production of the compositions. The optional active ingredients include, for example:
protein hydrolyzates,
vitamins,
anti-dandruff active ingredients,
carbohydrates,
bioquinones,
purines,
ectoin and/or
plant extracts.

Suitable protein hydrolyzates are understood to mean product mixtures that can be obtained by acid-, base-, or enzyme-catalyzed degradation of proteins. Protein hydrolyzates of plant, animal and/or marine origin can be used.

Animal protein hydrolyzates are, for example, elastin, collagen, keratin, silk and milk protein hydrolyzates, which can also be present in the form of salts. Such products are sold, for example, under the trade names Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Protein hydrolyzates of plant origin are preferred, for example soybean, almond, rice, pea, potato and wheat protein hydrolyzates. Such products are available, for example, under the trade names Gluadin® (Cognis), DiaMin® (Diamalt), Lexein® (Inolex) and Crotein® (Croda).

Cationized protein hydrolyzates can also be used, it being possible for the underlying protein hydrolyzate to originate from animals, for example from collagen, milk or keratin, from plants, for example from wheat, corn, rice, potatoes, soybeans or almonds, from marine life forms, for example from fish collagen or algae, or from biotechnologically obtained protein hydrolyzates.

The protein hydrolyzates underlying the cationic derivatives may be obtained from the corresponding proteins by chemical, in particular alkaline or acidic, hydrolysis, by enzymatic hydrolysis, and/or by a combination of both types of hydrolysis. The hydrolysis of proteins usually results in a protein hydrolyzate having a molecular weight distribution of approximately 100 daltons up to several thousand daltons. Cationic protein hydrolyzates of which the underlying protein portion has a molecular weight of from 100 to 25,000 daltons, preferably from 250 to 5,000 daltons, are preferred.

Furthermore, cationic protein hydrolyzates are understood to mean quaternized amino acids and mixtures thereof. Quaternization of the protein hydrolyzates or of the amino acids is often carried out by means of quaternary ammonium salts such as, for example, N,N-dimethyl-N-(n-alkyl)-N-(2-hydroxy-3-chloro-n-propyl)ammonium halides.

Furthermore, the cationic protein hydrolyzates can also be derivatized even further. Mentioned as typical examples of the cationic protein hydrolyzates and derivatives are the products that are commercially available and are known under the INCl names: cocodimonium hydroxypropyl hydrolyzed collagen, cocodimopnium hydroxypropyl hydrolyzed casein, cocodimonium hydroxypropyl hydrolyzed collagen, cocodimonium hydroxypropyl hydrolyzed hair keratin, cocodimonium hydroxypropyl hydrolyzed keratain, cocodimonium hydroxypropyl hydrolyzed rice protein, cocodimonium hydroxypropyl hydrolyzed silk, cocodimonium hydroxypropyl hydrolyzed soy protein, cocodimonium hydroxypropyl hydrolyzed wheat protein, cocodimonium hydroxypropyl silk amino acids, hydroxypropyl arginine lauryl/myristyl ether HCl, hydroxypropyltrimonium gelatin, hydroxypropyltrimonium hydrolyzed casein, hydroxypropyltrimonium hydrolyzed collagen, hydroxypropyltrimonium hydrolyzed conchiolin protein, hydroxypropyltrimonium hydrolyzed keratain, hydroxypropyltrimonium hydrolyzed rice bran protein, hydroxyproypltrimonium hydrolyzed silk, hydroxypropyltrimonium hydrolyzed soy protein, hydroxypropyl hydrolyzed vegetable protein, hydroxypropyltrimonium hydrolyzed wheat protein, hydroxypropyltrimonium hydrolyzed wheat protein/siloxysilicate, laurdimonium hydroxypropyl hydrolyzed soy protein, laurdimonium hydroxypropyl hydrolyzed wheat protein, laurdimonium hydroxypropyl hydrolyzed wheat protein/siloxysilicate, lauryldimonium hydroxypropyl hydrolyzed casein, lauryldimonium hydroxypropyl hydrolyzed collagen, lauryldimonium hydroxypropyl hydrolyzed keratain, lauryldimonium hydroxypropyl hydrolyzed silk, lauryldimonium hydroxypropyl hydrolyzed soy protein, steardimonium hydroxypropyl hydrolyzed casein, steardimonium hydroxypropyl hydrolyzed collagen, steardimonium hydroxypropyl hydrolyzed keratain, steardimonium hydroxypropyl hydrolyzed rice protein, steardimonium hydroxypropyl hydrolyzed silk, steardimonium hydroxypropyl hydrolyzed soy protein, steardimonium hydroxypropyl hydrolyzed vegetable protein, steardimonium hydroxypropyl hydrolyzed wheat protein, steartrimonium hydroxyethyl hydrolyzed collagen, quaternium-76 hydrolyzed collagen, quaternium-79 hydrolyzed collagen, quaternium-79 hydrolyzed keratain, quaternium-79 hydrolyzed milk protein, quaternium-79 hydrolyzed silk, quaternium-79 hydrolyzed soy protein, and quaternium-79 hydrolyzed wheat protein.

The weight proportion of the protein hydrolyzate(s) with respect to the total weight of the hair care compositions according to the invention can preferably be 0.01 to 5 wt. %, more preferably 0.025 to 3 wt. %, and in particular 0.05 to 2 wt. %.

Suitable vitamins are understood preferably to be the following vitamins, provitamins, and vitamin precursors, and derivatives thereof:
Vitamin A: the group of substances designated as vitamin A includes retinol (vitamin A₁) and 3,4-didehydroretinol (vitamin A₂). β-carotene is the provitamin of retinol. Suitable vitamin A components are, for example, vitamin A acid and esters thereof, vitamin A aldehyde, and vitamin A alcohol and esters thereof such as the palmitate and the acetate.
Vitamin B: the vitamin B group or the vitamin B complex includes, inter alia
   vitamin B₁ (thiamin)
   vitamin B₂ (riboflavin)
   vitamin B₃. The compounds nicotinic acid and nicotinamide (niacinamide) are often included under this designation.
   vitamin B₅ (panthothenic acid and panthenol). Panthenol is preferably used in the context of this group. Derivatives of panthenol which can be used are in particular the esters and ethers of panthenol, pantolactone and cationically derivatized panthenols. Individual representatives are, for example, panthenol triacetate, panthenol monoethyl ether, and the monoacetate thereof, as well as cationic panthenol derivatives.
   Vitamin B₆ (pyridoxine as well as pyridoxamine and pyridoxal).
Vitamin C (ascorbic acid): the use in the form of the palmitic acid ester, glucosides, or phosphates may be preferred. The use in combination with tocopherols may likewise be preferred.
Vitamin E (tocopherols, in particular α-tocopherol).
Vitamin F: the term “vitamin F” is usually understood to mean essential fatty acids, in particular linoleic acid, linolenic acid, and arachidonic acid.
Vitamin H: The compound (3aS,4S,6aR)-2-oxohexahydrothienol[3,4-d]imidazole-4-valeric acid is designated as vitamin H, however the trivial name biotin has become accepted for this compound.

Vitamins, provitamins, and vitamin precursors from the groups A, B, E, and H are particularly preferred. Nicotinamide, biotin, pantolactone, and/or panthenol are more particularly preferred.

The weight proportion of the vitamin(s), vitamin derivative(s), and/or the vitamin precursor(s) with respect to the total weight of the hair care compositions is preferably 0.001 to 2 wt. %, particularly preferably 0.005 to 1 wt. %, and in particular 0.01 to 0.5 wt. %.

Suitable anti-dandruff active ingredients can be selected from piroctone olamine, climbazole, zinc pyrithione, ketoconazoles, salicylic acid, sulfur, selenium sulfide, tar preparations, undecenoic acid derivatives, burdock extracts, poplar extracts, stinging nettle extracts, walnut shell extracts, birch extracts, willow bark extracts, rosemary extracts and/or arnica extracts. Climbazole, zinc pyrithione and piroctone olamine are preferred. Zinc pyrithione is particularly preferred.

The weight proportion of anti-dandruff agents with respect to the total weight of the hair care compositions can be preferably 0.01 to 10 wt. %, more preferably 0.025 to 7.5 wt. %, particularly preferably 0.05 to 5 wt. %, and in particular 0.075 to 3 wt. %.

Suitable carbohydrates can be selected from the group of monosaccharides and/or disaccharides and can preferably be used in an amount of from 0.01 to 5.00 wt. %, particularly preferably from 0.05 to 4.50 wt. % and in particular from 0.10 to 2.50 wt. % (based on the total weight of the composition) in the hair care compositions according to the invention.

Preferred monosaccharides and/or disaccharides are:
Monosaccharides such as D-ribose and/or D-xylose and/or L-arabinose and/or D-glucose and/or D-mannose and/or D-galactose and/or D-fructose and/or sorbose and/or L-fucose and/or L-rhamnose, and
Disaccharides such as sucrose and/or maltose and/or lactose and/or trehalose and/or cellobiose and/or gentiobiose and/or isomaltose.

In the compositions according to the invention, suitable bioquinones are to be understood to mean one or more ubiquinone(s) and/or plastoquinone(s). The ubiquinones preferred according to the invention have the following formula:

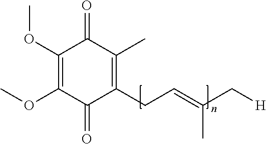

where n=6, 7, 8, 9 or 10.

The coenzyme Q-10 is most preferred here.

Bioquinones, in particular coenzyl Q-10, can preferably be used in the hair care compositions according to the invention (based on the total weight thereof) in an amount of from 0.001 to 1.0 wt. %.

Suitable purine and/or purine derivatives can preferably be used in the hair care compositions according to the invention (based on the total weight thereof) in an amount of from 0.001 to 2.5 wt. %. Cosmetic compositions which are preferred according to the invention are characterized in that they contain purine, adenine, guanine, uric acid, hypoxanthine, 6-purinethiol, 6-thioguanine, xanthine, caffeine, theobromine or theophylline. Caffeine is most preferred.

Ectoin ((S)-2-methyl-1,4,5,6-tetrahydro-4-pyrimidinecarboxylic acid can preferably be used in the hair care compositions according to the invention (based on the total weight thereof) in an amount of from 0.00001 to 1.0 wt. %.

By using plant extracts as care substances, the hair care compositions according to the invention can be formulated in a way that is in particular harmony with nature and is nevertheless very effective in terms of their care performance. It may optionally even be possible to omit the preservatives which are otherwise customary. According to the invention, particular preference is given to extracts of green tea, oak bark, stinging nettle, witch hazel, hops, henna, chamomile, burdock root, horsetail, hawthorn, lime blossom, almonds, aloe vera, spruce needles, horse chestnut, sandalwood, juniper, coconut, mango, apricot, lime, wheat, kiwi, melon, orange, grapefruit, sage, rosemary, birch, mallow, valerian, meadow foam flower, wild thyme, yarrow, thyme, lemon balm, restharrow, coltsfoot, marshmallow, meristem, ginseng, coffee, cocoa, moringa, ginger root and ayurvedic plant extracts such as, for example, Aegle marmelos (bilwa), Cyperus rotundus (nagar motha), Emblica officinalis (amalki), Morida citrifolia (ashyuka), Tinospora cordifolia (guduchi), Santalum album (chandana), Crocus sativus (kumkuma), Cinnamonum zeylanicum and Nelumbo nucifera (kamala), sweet grasses such as wheat, barley, rye, oats, spelt, maize, the various types of millet (sorghum, crabgrass, foxtail millet as examples), sugar cane, ryegrass, meadow foxtail, oat grass, bentgrass, meadow fescue, moor grass, bamboo, cotton grass, fountain grasses, Andropogonodeae (Imperata cylindrica, also called cogon grass), buffalo grass, cord grass, Bermuda grasses, love grass, Cymbopogon (lemongrass), Oryzeae (rice), Zizania (wild rice), marram grass, blue oat grass, creeping soft grass, quaking grasses, meadow grasses, couch grasses and Echinacea, in particular Echinacea purpurea (L.) Moench, all types of wine and perikarp of Litchi chinensis.

The plant extracts can be used both in pure and diluted form. If they are used in diluted form, they typically contain approximately 2 to 80 wt. % active substance and the extracting agent or extracting agent mixture used for their extraction as the solvent.

The cosmetic hair care compositions may also contain further active ingredients, auxiliaries, and additives, such as
UV filter substances,
structurants such as maleic acid and lactic acid,
swelling agents such as urea, allantoin, carbonates or hydantoin,
dyes for coloring the composition, this not being preferred according to the invention,
complexing agents such as EDTA, NTA, β-alanine diacetic acid, and phosphonic acids,
pearlescent agents such as ethylene glycol monostearate and distearate,
pigments,
propellants such as propane-butane mixtures, N₂O, dimethyl ether, CO₂ and air,
antioxidants,
perfume oils, perfumes and fragrances, these not being preferred according to the invention, provided that they are synthetic perfume oils, perfumes and fragrances.

The hair care compositions according to the invention preferably have a pH in the range from 2.5 to 5.0, preferably from 3.0 to 4.5, and in particular from 3.5 to 4.5.

Preferred hair care compositions according to the invention have a viscosity in the range from 3000 to 25,000 mPas, more preferably from 3,500 to 20,000 mPas, and in particular from 4,000 to 18,000 mPas (measured using Brookfield DV 2+; 2 rpm; spindle 5; 20° C.).

The hair care compositions according to the invention can be present in both single-phase and multi-phase form, for example in particular with two or three optically separated discrete phases. If the compositions are present in multi-phase form, the entire composition is converted to a single-phase composition by shaking prior to use and applied. After the shaking, spontaneous segregation occurs again and the discrete phases form again within about 1 minute up to 300 minutes. If the hair care compositions according to the invention are present in multi-phase form, they are preferably packaged in optically transparent packaging.

The compositions according to the invention can further be packaged as a pump spray, aerosol spray, pump foam or aerosol foam.

A second subject matter of the invention is the cosmetic use of the hair care composition according to the invention for improving the sensory properties as well as the resistance of hair to physical damage.

The statements made with respect to the compositions according to the invention apply, mutatis mutandis, to preferred embodiments for the use according to the invention.

The following examples are intended to explain the subject matter of the present invention, but in a non-limiting manner.

Patent examples: The following hair care compositions according to the invention were produced (the quantitative data refer to wt. %) and assessed as follows:

| | 1 | 2 | 3 | 4 | 5 |
|---|---|---|---|---|---|
| Betaine (trimethylgycine) | 0.1-2 | 0.5-1.5 | 0.5 | 1 | 1 |
| Esterquat according to | 0.01-6 | | | | |
| formula (II) | | | | | |
| Mono-, di-and | 0.01-3 | | | | |
| triglycerides of | | | | | |
| vegetable oils | | | | | |
| Stepanquat ® Soleil | | 0.05-8 | 3 | 1.5 | |
| (Dioleylethyl | | | | | |
| Hydroxyethylmonium | | | | | |
| Methosulfate, Sunflower | | | | | |
| Seed Oil Glycerides) | | | | | |
| Dehyquart ® F 75 T | | | | | 1.42 |
| (Distearoylethyl | | | | | |
| Hydroxyethylmonium | | | | | |
| Methosulfate, Cetearyl | | | | | |
| Alcohol) | | | | | |
| Amidoamine according | 0.1-3 | | | | |
| to formula (I) | | | | | |
| Pro Condition ® 22 | | 0.4-1 | 0.6 | 0.6 | 0.6 |
| (Brassicamidopropyl | | | | | |
| Dimethylamine) | | | | | |
| Triglycerides having a | 0.1-5 | | | | |
| chain length of 6 to 14 | | | | | |
| carbon atoms | | | | | |
| Caprylic/Capric | | 0.25-3 | 1 | | |
| Triglyceride | | | | | |
| Isopropyl ester having a | 0.1-5 | | | | |
| chain length of 10 to 20 | | | | | |
| carbon atoms | | | | | |
| Isopropyl myristate | | 0.25-3 | 0.8 | 0.8 | 0.8 |
| Vegetable oil | 0.01-3 | | | | |
| Argan oil | | 0.05-1 | 0.2 | | |
| Glycerol | 0.1-15 | 1-5 | 3 | | |
| Non-ionic emulsifier | 0.1-3 | | | | |
| Emulgade ® 1000 NI | | 0.25-1 | 0.5 | | |
| (Cetearyl Alcohol, | | | | | |
| Ceteareth-20) | | | | | |
| Fatty alcohol | 0.1-20 | | | | |
| Cetearyl alcohol | | 1-10 | 5 | 3.5 | 3.5 |
| Hydroxycarboxylic acid | 0.1-5 | | | | |
| Lactic Acid | | 0.4-2 | 0.6 | 0.6 | 0.6 |
| Optional further | 0-5 | | | | |
| auxiliaries and active | | | | | |
| ingredients | | | | | |
| Perfume, preservatives | | 0.1-3 | 1.6 | 1.6 | 1.6 |
| Water | up to | up to | up to | up to | up to |
| | 100 | 100 | 100 | 100 | 100 |

The compositions 4 (according to the invention) and 5 (comparative composition from the prior art) were analyzed and assessed in a single-blind test of 14 experts with respect to the hair care effect (feel). As a result, 10 of 14 experts rated the care effect of composition 4 as having a “better care effect;” one expert rated it as the same, and 3 experts preferred the comparative composition 5.

What is claimed is:

1. A silicone-free hair care composition comprising, based on the total weight of the hair care composition:
a) 0.1-2.0 wt. % trimethylglycine (INCl name: Betaine);
b) 0.05-8.0 wt. % of a mixture of
(b1) at least one esterquat of the following formula (II), 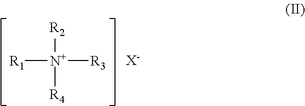
wherein
R₁, R₂ are identical or different and represent a C₁-C₄ alkyl radical,
R₃, R₄ are identical or different and represent the group —CH₂—CH(R₅)—OR₆, in which
R₅ represents a C₁-C₆ alkyl radical,
R₆ represents an acyl radical of an at least monounsaturated carboxylic acid having a C chain length of from 18 to 24 carbon atoms or represents the acyl radical of isostearic acid, and
X⁻ represents a halide, methyl sulfate, ethyl sulfate, maleate, fumarate, oxalate, tartrate, citrate, lactate or acetate ion, and
(b2) mono-, di- and triglycerides of vegetable oils; and
c) a cosmetic carrier.

2. The silicone-free hair care composition according to claim 1, wherein the esterquat (b1) is known under the INCl name Dioleylethyl Hydroxyethylmonium Methosulfate.

3. The silicone-free hair care composition according to claim 1, wherein the glyceride mixture (b2) is known under the INCl name Sunflower Seed Oil Glycerides.

4. The silicone-free hair care composition according to claim 1, further comprising at least one optionally cationized amidoamine in a weight proportion of 0.1-3.0 wt. % with respect to the total weight of the composition.

5. The silicone-free hair care composition according to claim 1, comprising an amidoamine of the following formula (I) 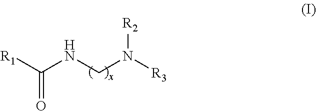
wherein
R₁ represents a saturated or unsaturated, branched or unbranched alk(en)yl radical having 19 to 30 carbon atoms,
R₂, R₃ represent, independently of one another, —H or a C₁-C₄ alkyl group, and
x represents an integer from 2 to 6.

6. The silicone-free hair care composition according to claim 4, wherein the amidoamine is known under the INCl name Brassicamidopropyl Dimethylamine.

7. The silicone-free hair care composition according to claim 1, comprising at least one further conditioning active ingredient selected from the group consisting of
triglycerides having a chain length of 6 to 14 carbon atoms,
isopropyl esters having a chain length of 10 to 20 carbon atoms,
vegetable oils,
glycerol, and
mixtures thereof.

8. The silicone-free hair care composition according to claim 1, comprising a non-ionic emulsifier in a weight proportion of up to 1.0 wt. % with respect to the total weight of the composition.

9. The silicone-free hair care composition according to claim 1, comprising at least one hydroxycarboxylic acid in a weight proportion of 0.1-5.0 wt. % with respect to the total weight of the composition.
